(19) United States
(12) Patent Application Publication
(10) Pub. No.: US 2007/0010541 A1
(43) Pub. Date: Jan. 11, 2007

(54) Methods for treating vascular diseases

(75) Inventors: Randall T. Peterson, Stoneham, MA (US); Calum A. Macrae, Newton Center, MA (US); Stanley Y. Shaw, Chestnut Hill, MA (US); David J. Millan, Boston, MA (US); Travis A. Peterson, Naperville, IL (US); Mark C. Fishman, Newton Center, MA (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 10/564,497

(22) PCT Filed: Jun. 30, 2004
(86) PCT No.: PCT/US04/20893

§ 371 Date: Sep. 8, 2006

(30) Foreign Application Priority Data
Jul. 14, 2003 (US) 60486964

(51) Int. Cl. A61K 31/44

(52) U.S. Cl. 514/277; 514/357

(57) ABSTRACT

The invention includes methods of treating or preventing vascular disease or promoting vascular growth or development. The methods can be used in the treatment of diseases associated with ischemia, such as heart attack and stroke.

### BACKGROUND OF THE INVENTION

This invention relates to methods of treating or preventing vascular disease or promoting vascular growth or development.

Angiogenesis and vasculogenesis are processes involved in the growth of blood vessels. Angiogenesis is the process by which new blood vessels are formed from extant capillaries, while vasculogenesis involves the growth of vessels from endothelial progenitor cells. Angiogenesis and vasculogenesis, and the factors that regulate these processes, are important in embryonic development, inflammation, and wound healing, and also contribute to pathologic conditions such as tumor growth, diabetic retinopathy, rheumatoid arthritis, and chronic inflammatory diseases (see, e.g., U.S. Pat. No. 5,318,957; Yancopoulos et al., Cell 93:661-664, 1998; Folkman et al., Cell 87:1153-1155, 1996; and Hanahan et al., Cell 86:353-364, 1996).

Both angiogenesis and vasculogenesis involve the proliferation of endothelial cells, which line the walls of blood vessels. The angiogenic process involves not only increased endothelial cell proliferation, but also includes a cascade of additional events, including protease secretion by endothelial cells, degradation of the basement membrane, migration through the surrounding matrix, proliferation, alignment, differentiation into tube-like structures, and synthesis of a new basement membrane. Vasculogenesis involves recruitment and differentiation of mesenchymal cells into angioblasts, which then differentiate into endothelial cells that form de novo vessels (see, e.g., Folkman et al., Cell 87:1153-1155, 1996).

Several angiogenic and/or vasculogenic agents with different properties and mechanisms of action are well known in the art. For example, acidic and basic fibroblast growth factor (FGF), transforming growth factor alpha (TGF-α) and beta (TGF-β), tumor necrosis factor (TNF), platelet-derived growth factor (PDGF), vascular endothelial cell growth factor (VEGF), and angiogenin are potent and well-characterized angiogenesis-promoting agents. In addition, both nitric oxide and prostaglandin have been shown to be mediators of various angiogenic factors, such as VEGF and bFGF.

Angiogenesis and vasculogenesis have been the focus of intense interest, as these processes can be exploited to therapeutic advantage. Stimulation of angiogenesis and/or vasculogenesis can aid in, for example, the healing of wounds, the vascularizing of grafts (e.g., skin grafts), and the enhancement of collateral circulation (e.g., in cases of vascular occlusion or stenosis). Stimulation of these processes can also be beneficial in treating or preventing ischemia, which occurs when a tissue does not receive an adequate supply of oxygen. Approaches to stimulate angiogenesis and/or vasculogenesis can involve the use or manipulation of the agents listed above, such as VEGF.

gridlock (grl) is an artery-restricted gene, expressed in the lateral posterior mesoderm, that guides the arterial-venous fate decision. Graded reduction of grl expression, by mutation or morpholino antisense oligonucleotides, progressively ablates regions of the artery and expands contiguous regions of the vein, preceded by an increase in expression of the venous markers and diminution of the expression of arterial markers. Zebrafish grl homozygotes exhibit a morphological defect of the dorsal aorta that prevents circulation to the trunk and tail, while circulation to the head is maintained. This phenotype is similar to the human congenital disorder aortic coarctation, a condition that affects nearly 1 in 1,000 live births and is a major source of morbidity and mortality in those affected. In mammalian cells, the gridlock gene has been shown to play roles in vasculogenesis, including the progression from endothelial cell proliferation and migration to vascular network formation. Mice lacking the gridlock gene exhibit ventricular septal defects, cardiomyopathies, and vascular defects.

### SUMMARY OF THE INVENTION

The invention provides methods of treating or preventing vascular diseases or promoting vascular growth or development in patients. The methods involve administration of compounds of formula (I):

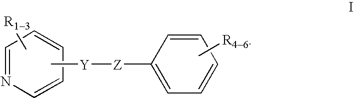

In formula (I), Y is selected from CH₂, C(CH₃), CH₂CH₂, CH₂CH₂CH₂, CH₂CH═CH, and CH₂CH C; Z is selected from S, SO, SO₂, O, and NR⁷; each of R₁, R₂, R₃, R₄, R₅, and R₆ are, independently, selected from H, halide, CF₃, C₁₋₃ alkyl, C₁₋₃ alkoxy, OH, SH, NO₂, CO₂H, SO₃H, and CN; and R⁷ is selected from H and C₁₋₃ alkyl. As specific examples, the compound of formula (I) can be selected from gs4012 and gs3999, as shown below.

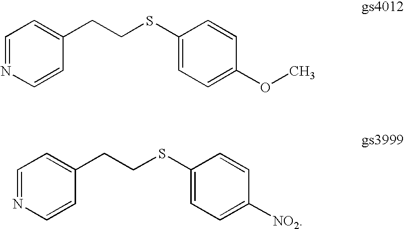

In one example of a method of the invention, a patient who has or is at risk of developing a disease or condition of the aorta, such as congenital dysplasia of the aorta (e.g., coarctation of the aorta) is treated using the compounds described herein. In another example, the vascular disease to be treated or prevented is ischemia. The tissues in which ischemia can occur, and which can be treated according to the invention, include, without limitation, muscle, brain, kidney, and lung. Ischemic diseases that can be treated or prevented using the methods of the invention include, for example, cerebrovascular ischemia, renal ischemia, pulmonary ischemia, limb ischemia, ischemic cardiomyopathy, and myocardial ischemia. The ischemia can result from, for example, a wound, vascular occlusion, or vascular stenosis.

The methods described herein can also be used to treat patients suffering or at risk of suffering a heart attack, stroke, or peripheral vascular disease. Further, the methods can be used for enhancing angiogenesis to accelerate wound healing, the vascularization of surgically transplanted tissue, or the healing of a surgically-created anastomosis.

Also included in the invention is the use of the compounds described herein in the treatment or prevention of diseases and conditions such as those noted herein, as well of the use of these compounds in the preparation of medicaments for these purposes. Further, the invention includes pharmaceutical kits and compositions including the compounds and, optionally, additional pharmacological agents and/or diluents.

The invention also includes pharmaceutical compositions that include one or more compounds of formula (I) and a pharmaceutically acceptable excipient. For example, the compositions can include gs4012 and/or gs3999.

Further, the invention includes methods for determining whether a candidate gene is a component of or affects a molecular pathway involved in vasculogenesis. These methods involve (i) treating a gridlock mutant embryo with a gridlock suppressor, and (ii) determining the effect of the suppressor on the expression of the gene. Detection of an altered level of expression of the gene, relative to the level in an untreated gridlock mutant embryo, indicates that the candidate gene is a component of or affects a molecular pathway involved in vasculogenesis. In these methods, the expression of the gene can be analyzed by, e.g., in situ hybridization or real-time polymerase chain reaction (PCR).

The invention further includes methods for identifying a gene in a molecular pathway involved in vasculogenesis, involving (i) treating a gridlock mutant embryo with a gridlock suppressor, (ii) extracting RNA from the treated embryo, (iii) reverse transcribing the extracted RNA into cDNA, (iv) contacting the cDNA with an array including an oligonucleotide library, and (v) identifying any genes corresponding to oligonucleotides of the array to which the cDNA from the treated embryo binds in a manner that is different from cDNA obtained from an untreated gridlock mutant embryo.

Further, the invention includes methods for identifying a component of a molecular pathway involved in vasculogenesis, involving (i) contacting a preparation including a candidate component (e.g., a lysate) with a matrix containing a gridlock suppressor, and (ii) identifying molecules that specifically bind to the gridlock suppressor.

In each of the methods described above, the gridlock suppressor used in the methods can be one of those described herein, another molecule within the formula provided herein, or other gridlock suppressors that can be identified, e.g., using the methods described herein.

In the generic descriptions of the compounds used in the invention, the number of atoms of a particular type in a substituent group is generally given as a range, e.g., an alkyl group containing from 1 to 3 carbon atoms or C₁₋₃ alkyl. Reference to such a range is intended to include specific references to groups having each of the integer number of atoms within the specified range. For example, an alkyl group from 1 to 3 carbon atoms includes each of C₁, C₂, and C₃. Other numbers of atoms and other types of atoms can be indicated in a similar manner.

As used herein, the terms “alkyl” and the prefix “alk-” are inclusive of both straight chain and branched chain groups and of cyclic groups. C₁₋₃ alkyl groups may be substituted or unsubstituted. Exemplary substituents include halide, hydroxyl, fluoroalkyl, perfluoralkyl, amino, aminoalkyl, disubstituted amino, and carboxyl groups. C₁₋₃ alkyls include, without limitation, methyl, ethyl, n-propyl, isopropyl, and cyclopropyl.

By “halide” is meant bromine, chlorine, iodine, or fluorine.

By “alkoxy” is meant a chemical substituent of the formula —OR, wherein R is selected from C₁₋₃ alkyl.

As used herein, a “pharmaceutical composition” refers to a formulation of a compound of formula (I) that would be suitable for approval by the Food and Drug Administration (FDA). The pharmaceutical composition could be suitable for approval if the composition meets efficacy and toxicity standards established by the FDA. The pharmaceutical composition can be shown to meet those standards using established methods of testing which are acceptable to the FDA. To reduce the toxicity of the pharmaceutical composition, each of the components of the composition are required to meet standards of purity. Thus, the compound of formula (I) will be purified to remove reaction side-products (e.g., any product formed during the synthesis of the compound of formula (I) which is not the desired compound) and reaction residues (e.g., reaction solvents, reagents, and salts) prior to the formulation of the pharmaceutical composition. For the pharmaceutical compositions described herein, the reaction side-products and residues are generally less than 2%, 1%, 0.5%, or 0.1% (w/w) of the mass of the compound of formula (I) used in the pharmaceutical formulation.

As used herein, the term “treating” refers to administering a pharmaceutical composition for prophylactic and/or therapeutic purposes. To “prevent disease” refers to prophylactic treatment of a human patient who is not yet ill, but who is susceptible to, or otherwise at risk of, a particular disease. To “treat disease” or use for “therapeutic treatment” refers to administering treatment to a patient already suffering from a disease to improve or stabilize the patient's condition. Thus, in the claims and embodiments, treating is the administration to a human patient either for therapeutic or prophylactic purposes.

As used herein, the term “administration” or “administering” refers to a method of giving a dosage of a pharmaceutical composition to a patient, where the method is, e.g., topical, oral, intravenous, intraperitoneal, or intramuscular (see below). The method of administration selected can vary depending on various factors, e.g., the components of the pharmaceutical composition, the site of the potential or actual disease, and the severity of disease.

Other features of the invention will be apparent from the detailed description of the invention, the drawings, and the claims.

### BRIEF DESCRIPTION OF THE DRAWINGS

FIG. 1A is a microangiogram of an untreated grl−/− embryo 48 hours postfertilization.

FIG. 1B is an microangiogram of a grl−/− embryo 48 hours postfertilization treated with the small molecule gs4012 beginning 6 hours postfertilization.

FIG. 1C is a diagram depicting the chemical structures of two small molecules, gs4012 (top) and gs3999 (bottom), identified as gridlock suppressors by whole-organism chemical screening.

FIG. 1D is a graph depicting a dose response curve for suppression of the gridlock mutant phenotype by gs4012.

FIGS. 1E and 1F are pictures depicting 6 micron sections comparing the histology of the aorta in grl−/− embryos left untreated (FIG. 1E) or treated with 2.5 μg/mL gs4012 (FIG. 1F).

FIG. 2A is a graph depicting the rescue percentage versus the timing of gridlock suppression by gs4012, as a function of when gs4012 is administered (closed diamonds), and as a function of when gs4012, added during gastrulation, is washed away (open squares). Each time point represents the difference between the percentages of wild-type circulation observed in treated and untreated embryos.

FIG. 2B depicts the effect of gs4012 on expression of several genes involved in formation of the aorta using quantitative real-time PCR (RT-PCR).

FIG. 2C is a graph depicting the expression levels of gridlock (closed diamonds) and VEGF (open squares) mRNA during development.

FIG. 3A is a bar graph showing the percentage of grl−/− embryos exhibiting circulation to the tail that are not injected (NI), injected with empty plasmid (pCS2), or with plasmid containing the murine VEGF cDNA (VEGF).

FIG. 3B is a microangiogram of a grl−/− embryo not treated.

FIG. 3C is a microangiogram of a grl−/− embryo injected with pmVEGF (assessed 48 hpf).

### DETAILED DESCRIPTION

The invention provides methods of treating or preventing vascular disease or promoting vascular growth or development by administering the compounds described herein. These methods can be useful, for example, in the treatment of diseases associated with ischemia. Also included in the invention are pharmaceutical compositions including the compounds described herein. The methods and compositions of the invention are described further, as follows.

Therapy

The methods and pharmaceutical compositions of the invention can be used to promote vascular growth for the treatment or prevention of conditions associated with ischemia. Such conditions include, for example, stroke or heart attack. In addition, the methods and compositions can be used to accelerate wound healing, to promote vascularization of surgically transplanted tissue, and to enhance the healing of a surgically-created anastomosis.

The methods and compositions of the invention can also be used in the treatment of dysplasias of the aorta. In particular, the aorta, which is the main trunk of the systemic arterial network, is subject to several congenital and acquired disorders that can lead to severe complications in infancy and adulthood. Coarctation of the aorta is one of the most common human congenital cardiovascular diseases. In coarctation, a discrete, localized vascular malformation partially obstructs the descending aorta, the major artery to the body, and most frequently occurs distal to the origins of vessels supplying the head and arms. Its effects often become more physiologically severe at birth, when closure of the ductus can exacerbate the restriction to aortic blood flow. As a consequence of coarctation, affected individuals suffer from high blood pressure in the upper extremities and head, and from low pressure in the trunk and legs. Survival often depends on the development of collateral blood vessels, which facilitate blood circulation in a manner so as to bypass the lesion. The methods and compositions of the invention thus can be used to treat patients with coarctation of the aorta, as well as other aortic diseases, such as interrupted arch disease.

A patient can be suffering from or be at risk of suffering from ischemic damage when one or more tissues within the patient are deprived of an adequate supply of oxygenated blood. The interruption of the supply of oxygenated blood is often caused by a vascular occlusion. Such vascular occlusion can be caused by, for example, arteriosclerosis, trauma, or surgical procedures. There are many ways to determine if a tissue is at risk of suffering ischemic damage from an undesirable vascular occlusion. Such methods are well known to physicians who treat such conditions. For example, in myocardial disease, these methods can include a variety of imaging techniques (e.g., radiotracer methodologies, such as ⁹⁹mTc-sestamibi, x-ray, and MRI angiography) and physiological tests. The induction of angiogenesis in tissue affected by or at risk of being affected by a vascular occlusion, using the methods and compositions of the invention, can be an effective means of preventing and/or attenuating ischemia in such tissue.

In ischemic conditions, the blood supply to discrete organs, such as the brain, heart, pancreas, or limbs, can be attenuated by disease, trauma, surgery, or other events. The alleviation of such attenuated blood supply regardless of its origin is included in the present invention. Thus, prevention or alleviation of damage from indications such as myocardial ischemia and stroke are included. Additionally, the planning of a surgical procedure can be predictive of the interruption of blood supply through a particular portion of a patient's vasculature. Prior or concurrent treatment according to the present invention can substantially improve the outcome of these surgeries.

Synthesis

Compounds of formula (I) can be prepared using commercially available starting materials and established synthetic protocols. Numerous methods are available for the synthesis of compounds of formula (I). For example, compounds of formula (I) can be prepared as described in Scheme 1 using alkylthio-de-halogenation, alkoxy-de-halogenation, or amino-de-halogenation under basic conditions. See, for example, J. March, Advanced Organic Chemistry: Reactions, Mechanisms and Structure, John Wiley & Sons, Inc. pp. 654-656, 1992. In the schemes, n is an integer, R¹⁰ is as defined aboved, and Rₙ is one or more ring substitutions. The requisite hydroxy, thio, or amino pyridine derivative can be obtained, for example, from the corresponding halide (e.g., by substitution reaction) or nitrile (e.g., by reduction to an amine) using synthetic methods familiar in the art. Some compounds are more easily prepared by reductive amination, as shown in Scheme 2. For the appropriate conditions, see J. March, Advanced Organic Chemistry: Reactions, Mechanisms and Structure, John Wiley & Sons, Inc. pp. 898-901, 1992, and references cited therein. Commercially available starting materials useful in the synthesis of compounds of formula (I) include 2,3,5,6-Tetrafluoro-4-pyridinecarbonitrile (Aldrich Cat. No. 34,459-1); 2,6-Dihydroxy-4-methyl-3-pyridinecarbonitrile (Aldrich Cat. No. 37,947-6); 3-(2-Aminoethyl)pyridine Dihydrobromide (Aldrich Cat. No. 51,811-5); 3-(Bromomethyl)pyridine hydrobromide (Aldrich Cat. No. 47,551-3); 3-(Aminomethyl)pyridine (Aldrich Cat. No. A6,540-9); 3-Pyridinecarboxaldehyde (Aldrich Cat. No. P6,220-8); 4-(2-Aminoethyl)pyridine (Aldrich Cat. No. 21,624-0); 4-(Bromomethyl)pyridine hydrobromide (Aldrich Cat. No. 49,174-8); 4-(Ethylaminomethyl)pyridine (Aldrich Cat. No. 36,709-5); 4-Pyridinecarboxaldehyde (Aldrich Cat. No. P6,240-2); 4-Aminomethyl-5-hydroxy-6-methyl-3-pyridinemethanol (Aldrich Cat. No. P9158); 3-Acetylpyridine (Aldrich Cat. No. A2,120-7); 4-Acetylpyridine (Aldrich Cat. No. A2,140-1); 3-Pyridinecarboxaldehyde (Aldrich Cat. No. P6,220-8); 2-Methyl-3-hydroxymethylpyridine (Aldrich Cat. No. 53,510-9); 3-Pyridinemethanol (Aldrich Cat. No. P6,680-7); 2,6-Dichloro-3-(2-chloroethyl)-4-methylpyridine (Aldrich Cat. No. S26,611-6); and 2-Chloro-5-(chloromethyl)pyridine (Aldrich Cat. No. 51,691-0).

Numerous substituted aryl bromides (i.e., for use in reaction Scheme 1) and anilines (for use in reaction Scheme 2) are commercially available or are readily synthesized from commercially available starting material using standard synthetic techniques. Using the approach described in Scheme 1, gs4012 and gs3999 can be prepared by the reaction of 4-(2-mercaptylethyl)pyridine with 4-methoxy bromobenzene or 4-nitro bromobenzene, respectively.

Sulfoxides and sulfones can be prepared by the oxidation of the corresponding thioether as shown in Scheme 3. For the appropriate conditions, see J. March, Advanced Organic Chemistry: Reactions, Mechanisms and Structure, John Wiley & Sons, Inc. pp. 1201-1202, 1992.

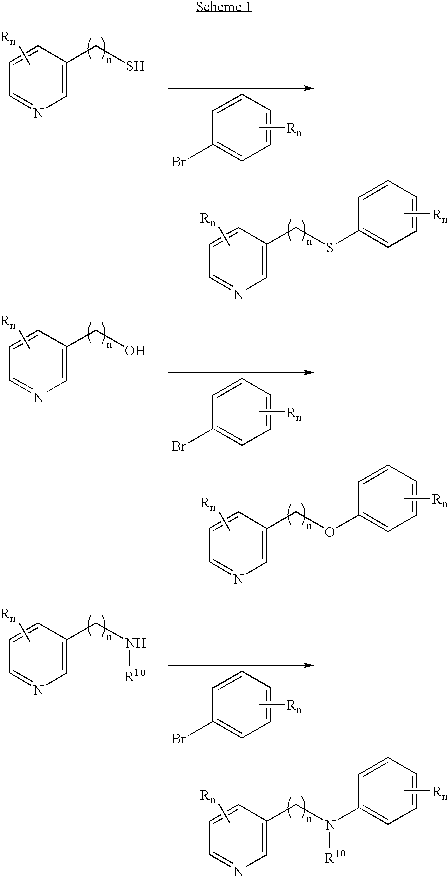

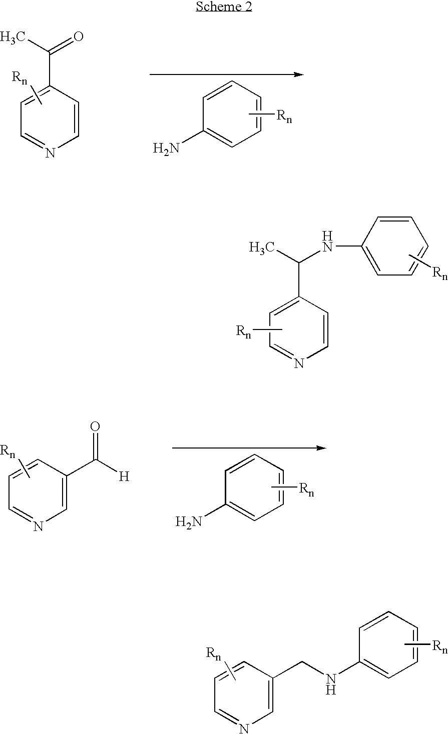

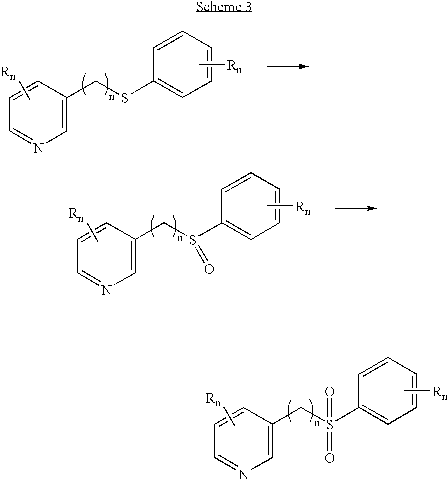

In some instances, the synthesis will require the selective protection and deprotection of alcohols, amines, sulfhydryls, and/or carboxylic acid functional groups. This can be achieved using known techniques. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxylic acids include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxylic acid functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994 (incorporated herein by reference).

Formulation and Administration

The pharmaceutical compositions of the invention can be administered by any appropriate route for promoting vascular growth or for the treatment or prevention of diseases or conditions associated with ischemia. They can be administered to humans, domestic pets, livestock, or other animals with a pharmaceutically acceptable diluent, carrier, or excipient, in unit dosage form. Administration can be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, oral, or by suppository.

The exemplary dosages of the pharmaceutical compositions described herein to be administered will depend on such variables as the type and extent of the disorder, the overall health status of the patient, the therapeutic index of the selected compound of formula (I), and the route of administration. Standard clinical trials can be used to optimize the dose and dosing frequency for any particular pharmaceutical composition of the invention.

Therapeutic formulations can be in the form of liquid solutions or suspensions; for oral administration, formulations can be in the form of tablets or capsules; and for intranasal formulations, in the form of powders, nasal drops, or aerosols.

Methods for making formulations are well known in the art and can be found, for example, in Remington: The Science and Practice of Pharmacy (20^{th} ed., ed. A. R. Gennaro AR.), Lippincott Williams & Wilkins, 2000. Formulations for parenteral administration can contain, for example, excipients, sterile water, or saline; polyalkylene glycols such as polyethylene glycol; oils of vegetable origin; or hydrogenated napthalenes. Biocompatible, biodegradable lactide polymer, lactide/glycolide copolymer, or polyoxyethylene-polyoxypropylene copolymers can be used to control the release of the compounds. Nanoparticulate formulations (e.g., biodegradable nanoparticles, solid lipid nanoparticles, liposomes) can be used to control the biodistribution of the compounds. Other parenteral delivery systems that can be used in the invention include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

The compound of formula (I) can optionally be administered as a pharmaceutically acceptable salt, such as a non-toxic acid addition salt or a metal complex that is commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid, phosphoric acid, or the like. Metal complexes include zinc, iron, and the like.

Controlled release formulations are useful where the compound of formula (I), has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the therapeutic compound. For example, controlled release can be obtained by the appropriate selection of formulation parameters and ingredients (e.g., appropriate controlled release compositions and coatings). Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients can be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use can also be provided as chewable tablets, or as hard gelatin capsules in which the active ingredient is mixed with an inert solid diluent, or as soft gelatin capsules in which the active ingredient is mixed with water or an oil medium.

In addition to being administered to patients in therapeutic methods, the compounds described herein can also be used to treat cells and tissues ex vivo. For example, the compounds can be used to treat explanted tissues that may be used, for example, in transplantation.

Experimental Use

The invention also includes the use of the compounds described herein as research tools. For example, the compounds can be used in the development of other compounds having activities such as those described herein. In these methods, the compounds described herein can be used as models for the design of further compounds or as controls in testing other compounds. Further, the invention includes use of the compounds described herein (or other gridlock suppressors) in the study of the processes of vasculogenesis and angiogenesis. For example, the compounds can be used in the identification of the molecular targets of the gridlock suppressors, which will help enumerate the molecular participants in the process of vasculogenesis. Furthermore, the suppressors can be used as tools for dissecting the mechanisms by which gridlock and its suppressors contribute to formation of the major vessels. Just as identification of a genetic suppressor helps piece together the relationships between genes in a pathway, characterization of these chemical suppressors can be used to clarify gridlock's role in vasculogenesis and related processes. These methods can include, for example, (i) candidate gene analysis, (ii) expression profiling, and (iii) direct biochemical purification of small molecule targets. Each of these methods is described further below.

Candidate Gene Analysis

Several genes are believed to be involved in the process of vasculogenesis. These include notch1a, notch5, sonic hedgehog, vegf, flk1, flt4, EphrinB2a, and EphB4, among others. The effects gridlock suppressors, such as those described above, on the expression of these and other candidate genes can be tested. Both the pattern of expression and the level of expression can be determined, using, for example, whole mount in situ hybridization and real-time polymerase chain reaction (PCR), respectively.

In the case of whole mount in situ hybridization, grl−/− embryos can be treated with the gridlock suppressors, collected at various time points, and fixed with paraformaldeyde. In situ hybridization can then be performed using a panel of probes specific for the genes listed above and other vascular probes. This in situ hybridization protocol is amenable to high-throughput analysis.

In the case of real-time PCR, groups of 20 grl−/− embryos are left untreated or treated with a gridlock suppressor. At 24 hours post fertilization, total RNA can be extracted using the Trizol method. Equivalent amounts of total RNA from each sample can be reverse transcribed into cDNA. This cDNA can serve as template for real-time PCR to determine the relative expression quantities for the genes listed above. RT-PCR can be performed with an Applied Biosystems Sequence Detection System 7000. Taqman probes and primers can be designed using zebrafish genomic sequences made publicly available by the Wellcome Trust Sanger Institute.

Expression Profiling

To determine the involvement in gridlock suppression of genes not listed as candidate genes, expression profiling can be performed on embryos treated with the gridlock suppressors. The goal of such studies is to identify novel pathways that are affected by the small molecule gridlock suppressors that may be involved in vasculogenesis.

In an example of this approach, zebrafish oligonucleotide arrays are produced by printing a set of 14,000 zebrafish oligonucleotides previously purchased from MWG Biotech onto Corning CGAP glass slides using a GeneMachines OmniGrid arrayer. For each condition, 50 zebrafish embryos are treated or left untreated for the desired time before total RNA is extracted using the Trizol method. 20 μg total RNA from each sample is reverse transcribed to cDNA using a dNTP mix containing aminoallyl dUTP. The remaining RNA is hydrolyzed and the cDNA is fluorescently labeled with Cy3 or Cy5 by direct reaction with the aminoallyl-dUTP. Labeled cDNAs are hybridized to the oligonucleotide array under a glass coverslip for 14-18 hours at 65 C. The slides are then washed, dried, and scanned using an Axon 4000 scanner. Data are interpreted using GenePix software, and clustering of data sets from the various compounds is performed using Rosetta Resolver software.

Biochemical Purification of Small Molecule Targets

A very direct means of determining the mechanism of gridlock suppression is biochemical purification of the proteins targeted by the suppressors. The gridlock suppressors can be covalently attached to solid support beads via linkers, such as alkyl linkers, and the resulting affinity matrices can be used for chromatographic purification of target proteins. The identities of the purified targets can be determined by tandem mass spectrometry. This approach offers the potential for completely unbiased discovery of novel pathways that affect vasculogenesis.

For example, prior to affinity chromatography, a site for attachment of a chemical linker must be identified. Variants of the compound containing modifications at various potential linker attachment points can be purchased or synthesized, and then tested for efficacy and potency. Once an attachment point is identified that does not interfere with a compound's activity, a linker (e.g., aminocaproic acid) is appended to the compound. The linker is then covalently attached to Affigel (Biorad) solid support resin following the manufacturer's instructions. A negative control matrix is also synthesized by attaching the linker to the compound of interest at an inactivating site, when possible, or by attaching linker alone to the Affigel.

By performing a time course experiment, a narrow developmental window can be identified during which a compound must be present to suppress the gridlock phenotype. About 500 zebrafish embryos of the appropriate developmental stage are dechorionated manually and lysed by douncing in 1 mL of phosphate-buffered saline (PBS) containing 1% Triton X100 detergent. The lysate is cleared by centrifugation and loaded onto microcolumns containing the affinity matrix or a control matrix. The column is rinsed with 2 mL of PBS with 1% Triton X100 and then eluted with 1 mL, of PBS with 1% Triton X100 containing 10 μM free compound. Column fractions of 0.25 mL are collected, precipitated with trichloroacetic acid, and subjected to sodium dodecyl sulfate polyacrylamide gel electrophoresis. The acrylamide gels are fixed and silver stained. Protein bands present in fractions from the affinity column but not the control column are excised and submitted for LC MS/MS sequencing.

Identified targets can be confirmed to be involved in gridlock suppression by performing gene knockdowns of the identified targets using antisense morpholino oligonucleotide injections. Therefore, even if multiple binding partners exist, it is possible to identify which ones are relevant to gridlock suppression.

Experimental Results

The following is a description of experiments showing the feasibility of using a whole organism, phenotype-based small molecule screen to discover candidate curative molecules for a common congenital cardiovascular malformation. The zebrafish mutation, gridlock, disrupts the bifurcation of the aorta, blocking distal blood flow in a region and physiological manner akin to aortic coarctation in humans (see Lawson et al., Development 128(19):3675-3683, 2001; Zhong et al., Science 287(5459):1820-1824, 2000; Weinstein et al., Nat. Med. 1(11):1143-1147, 1995; Towbin et al., Nat. Med. 1(11):1141-1142, 1995). We identified through unbiased chemical screening a class of compounds that suppress the coarctation phenotype and permit survival to adulthood. These compounds function during specification and migration of angioblasts. They act to upregulate expression of vascular endothelial growth factor (VEGF), and the activation of the VEGF pathway is sufficient to suppress the grl phenotype. Thus, novel small molecules can be discovered that ameliorate complex dysmorphic syndromes even without targeting the affected gene directly.

Genetic suppressor screens can identify second-site mutations that modify the effect of an existing genetic mutation. This approach identifies by phenotypic rescue components of pathways, and parallel pathways, otherwise difficult to identify by biochemical means (St. Johnston, Nat. Rev. Genet. 3(3):176-188, 2002). We reasoned that a screen for chemical suppressors of a genetic mutation, carried out in a vertebrate model organism, might: (a) provide insights into vertebrate-specific processes, (b) permit temporal dissection of their timing, and (c) provide small molecule therapeutics for the treatment of aortic arch and other diseases.

To test the feasibility of this approach, we screened for chemical suppressors of the zebrafish gridlock mutation. Gridlock (grl, also Hey2, HRT2, CHF1, HERP1, HESR2) is a bHLH transcriptional repressor (Zhong et al., Science 287(5459):1820-1824, 2000). In zebrafish, grl−/− mutants, which have hypomorphic mutations in the grl gene, suffer from a malformed aorta that prevents circulation to the trunk and tail (Zhong et al., Science 287(5459):1820-1824, 2000; Weinstein et al., Nat. Med. 1(11):1143-1147, 1995). As such, these mutants have been proposed as a model of coarctation of the aorta, a common cardiovascular malformation in humans (Towbin et al., Nat. Med. 1(11):1141-2, 1995).

In the mouse, null mutations of the grl/Hey2 gene cause deformities of the major vessels and also defects of the heart (Sakata et al., Proc. Natl. Acad. Sci. U.S.A. 99(25):16197-202, 2002; Donovan et al., Curr. Biol. 12(18):1605-1610, 2002; Gessler et al., Curr. Biol. 12(18):1601-1604, 2002).

Identification of Compounds that Suppress the Gridlock Phenotype in grl−/− Embryos

We arrayed grl−/− embryos in 96-well plates, exposed them to small molecules from a structurally-diverse chemical library, and examined their circulation after 48 hours of development. The compounds were purchased as 5 mg/mL stock solutions in DMSO from Cambridge Chemical.

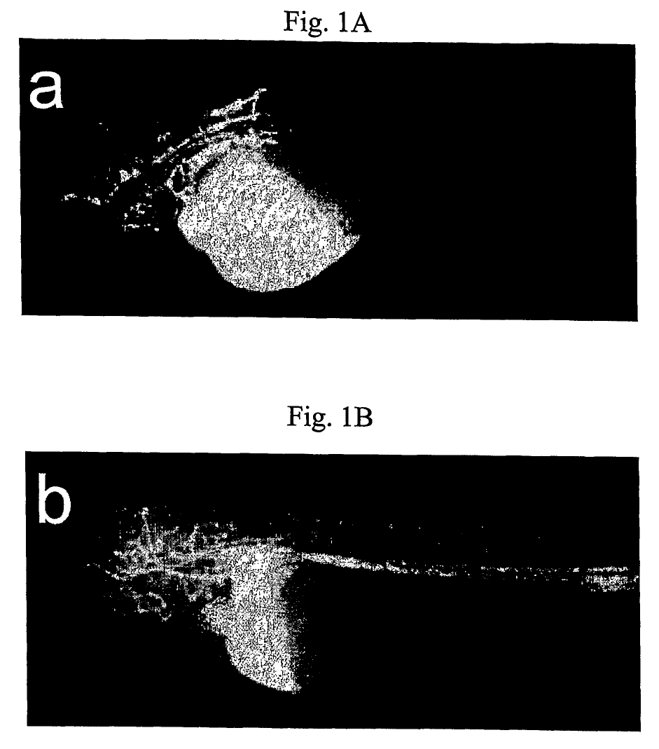

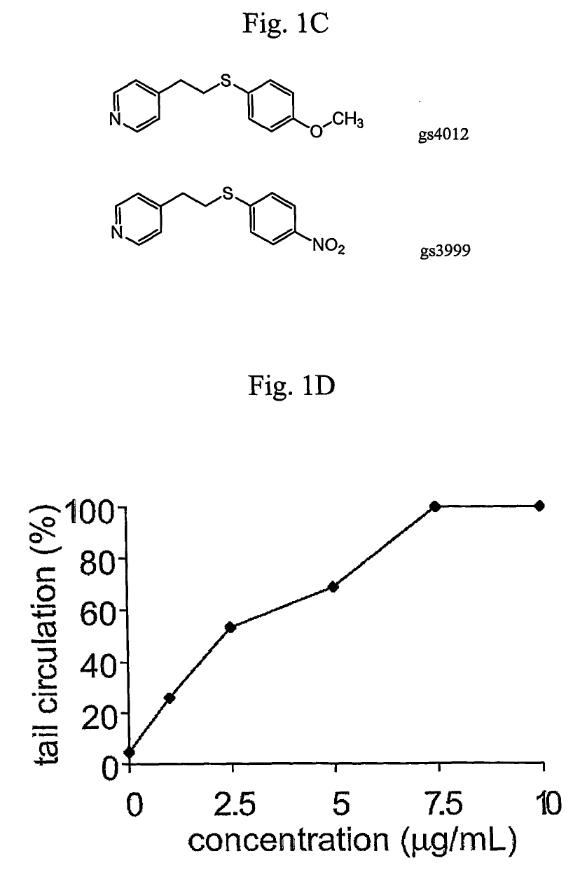

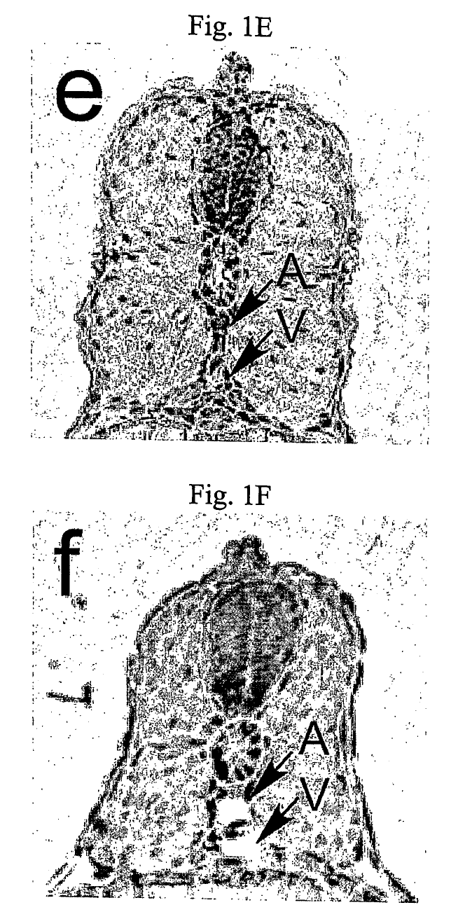

Two of 5,000 small molecules examined suppress the gridlock phenotype in grl−/− embryos, restoring circulation to the tail (FIG. 1B). We confirmed the functional restoration of the aorta by microangiography (FIGS. 1A-B) and histology (FIGS. 1E-F). The suppressors are structurally-related, sulfur-containing compounds (FIG. 1C) that exhibit this rescue in a dose-dependent manner (FIG. 1D). The compound gs4012 is a more potent suppressor than gs3999, and so it was used for all subsequent experiments.

Suppression of the Gridlock Phenotype as a Function of Developmental Stage

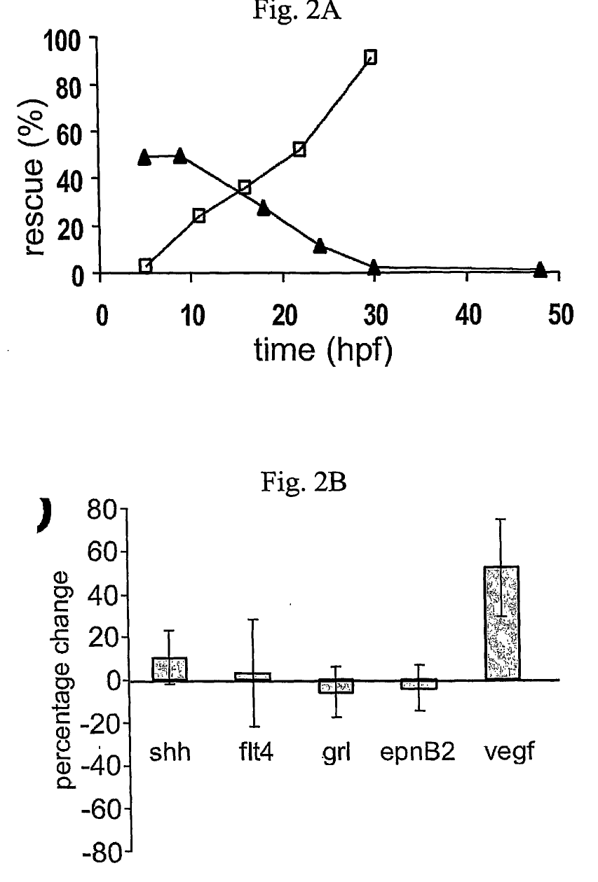

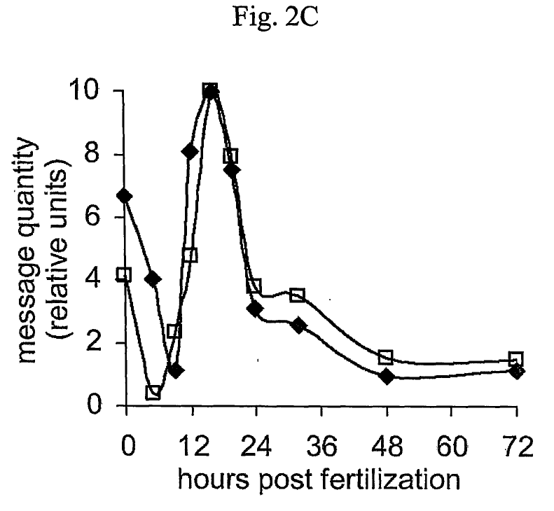

Angioblasts in zebrafish are first evident around 14 hours postfertilization (hpf) and begin to aggregate at the midline by 16 hpf (Eriksson et al., J. Morphol. 244(3):167-176, 2000; Fouquet et al., Dev. Biol. 183(1):37-48, 1997). The lumen of the aorta begins to form in the posterior trunk at 17.5 hpf, but is not complete until just prior to the initiation of circulation 24 hpf. The critical period for gs4012 suppression is between 12 and 24 hpf (FIG. 2A). Little or no suppression is observed when treatment begins after 24 hpf.

Similarly, when gs4012 is added during gastrulation and washed away at later and later timepoints, the effectiveness of suppression is increased, reaching maximal suppression by 30 hpf. Suppression is maintained after removal of the suppressor, and rescued mutants remain viable to adulthood. Therefore, suppression is irreversible and largely complete prior to actual formation of the aorta. Furthermore, the suppressors' activity is required precisely during the period of angioblast cell fate determination and migration, suggesting a direct role in these processes.

We used real-time PCR to quantitate the expression of several genes believed to play roles in angioblast cell fate determination or migration. One of these genes, VEGF, is upregulated 24 hpf by exposure to gs4012 (FIG. 2B). VEGF plays an important role in formation of the aorta. VEGF122 expression in the Xenopus hypochord acts as a chemoattractant, stimulating angioblast migration to the midline prior to aorta formation (Cleaver et al., Development 125(19):3905-3914, 1998). Targeted disruption of VEGF in mice causes severely disrupted aortic development (Carmeliet et al., Nature 380(6573):435-439, 1996; Ferrara et al., Nature 380(6573):439-442, 1996), and a VEGF promoter haplotype is associated with an increased risk for cardiovascular defects (including coarctation of the aorta) in humans with deletions in the DiGeorge locus 22q11 (Stalmans et al., Nat. Med. 9(2):173-182, 2003). In zebrafish, gridlock and VEGF transcript levels are temporally correlated throughout development (FIG. 2C). Both genes are upregulated between 10 and 24 hpf, the period of greatest sensitivity to gridlock suppression, and are expressed in adjacent tissues during this period.

The Effect of Increased VEGF Expression Upon Suppression of the Gridlock Phenotype

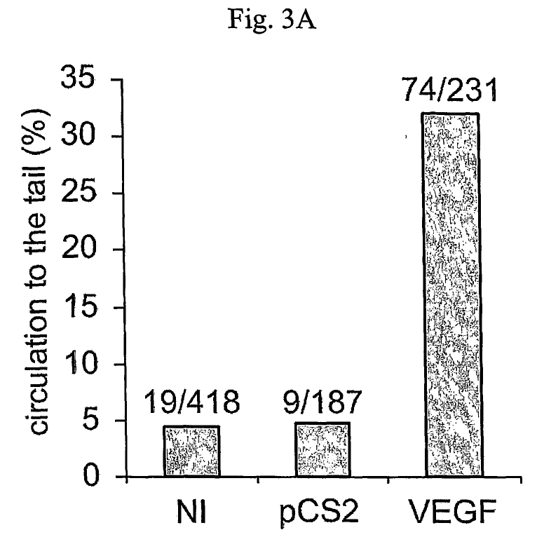

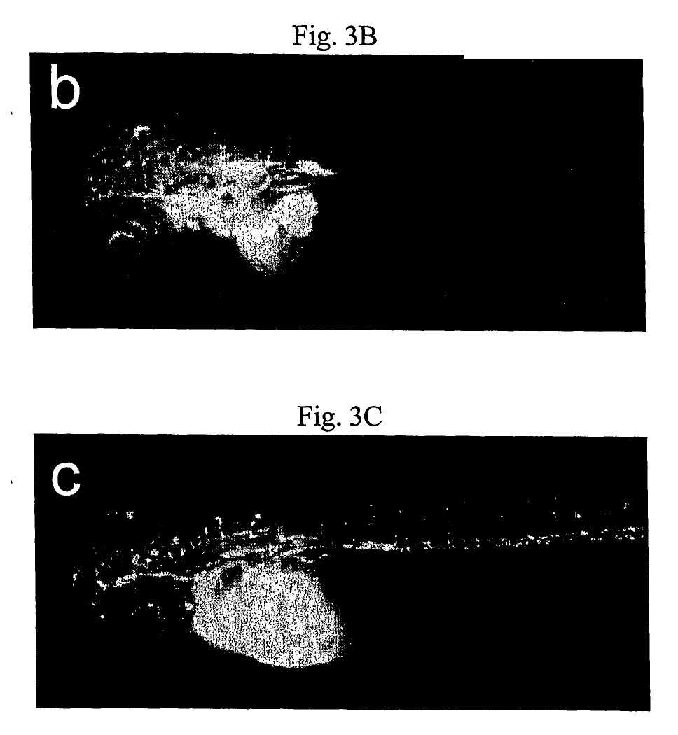

To test whether the increased VEGF expression induced by gs4012 could be responsible for suppression of the gridlock phenotype, we injected grl−/− embryos with an expression plasmid containing the murine VEGF cDNA (pmVEGF). About one third of the mutant embryos injected with pmVEGF exhibited wild-type circulation to the trunk and tail, whereas only about 5 percent of uninjected or vector-injected embryos exhibited wild-type circulation (FIG. 3). Therefore, VEGF overexpression is sufficient to suppress the mutant phenotype in grl−/− embryos. Chemical suppression of the gridlock phenotype appears to be more efficient than injection of pmVEGF, perhaps because plasmid-driven VEGF expression is mosaic or because the chemical suppressors have additional mechanisms of action beyond VEGF induction.

Gridlock is a transcriptional repressor that drives angioblast differentiation to an arterial fate rather than a venous fate (Zhong et al., Nature 414(6860):216-220, 2001). It appears to function downstream of Notch in this bimodal cell fate decision (Zhong et al., Nature 414(6860):216-220, 2001; Iso et al., J. Biol. Chem. 277(8):6598-6607, 2002; Nakagawa et al., Proc. Natl. Acad. Sci. U.S.A. 97(25):13655-13660, 2000; Iso et al., Mol. Cell Biol. 21(17):6080-6089, 2001). VEGF acts to expand the number of angioblasts and to draw them towards the midline during vessel formation (Cleaver et al., Development 125(19):3905-3914, 1998; Ash et al., Dev. Biol. 223(2):383-398, 2000). Thus, we presume that the rescue by VEGF-inducing gs4012 treatment or by VEGF cDNA injection reflects stimulation of these compensatory processes. Regardless of the exact nature of the relationship between gridlock and VEGF, it is clear that they are related functionally; overexpression of one compensates for hypomorphic mutation of the other. This relationship was not previously identified, illustrating one of the benefits of unbiased screening for suppressors of a genetic mutation, namely identifying novel interactions between biological pathways.

### OTHER EMBODIMENTS

All publications and patent applications mentioned in this specification are incorporated herein by reference to the same extent as if each independent publication or patent application was specifically and individually indicated to be incorporated by reference.

While the invention has been described in connection with specific embodiments thereof, it is to be understood that it is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the appended claims.

What is claimed is:

1. A method of treating or preventing vascular disease or promoting vascular growth or development in a patient, said method comprising administering to the patient a compound of formula (I): 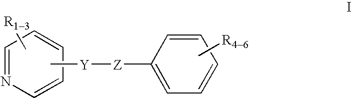
wherein,
Y is selected from CH₂, C(CH₃), CH₂CH₂, CH₂CH₂CH₂, CH₂CH═CH, and CH₂CH C;
Z is selected from S, SO, SO₂, O, and NR⁷;
each of R₁, R₂, R₃, R₄, R₅, and R₆ are, independently, selected from H, halide, CF₃, C₁₋₃ alkyl, C₁₋₃ alkoxy, OH, SH NO₂, CO₂H, SO₃H, and CN; and
R⁷ is selected from H and C₁₋₃ alkyl.

2. The method of claim 1, wherein the structure of said compound is selected from:

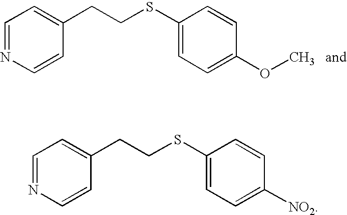

3. The method of claim 1, wherein said patient has or is at risk of developing a disease or condition of the aorta.

4. The method of claim 3, wherein said disease or condition is congenital dysplasia of the aorta.

5. The method of claim 4, wherein said congenital dysplasia of the aorta is coarctation of the aorta.

6. The method of claim 1, wherein said patient has or is at risk of developing ischemia.

7. The method of claim 6, wherein said ischemia is myocardial, cerebral, mesenteric, or limb ischemia; said ischemia results from a wound, vascular occlusion, or vascular stenosis; or said patient has suffered or is at risk of suffering a heart attack or stroke.

8. The method of claim 1, wherein said patient has peripheral vascular disease.

9. The method of claim 1, wherein said vascular growth or development is at the site of a wound in said patient; in a tissue that has been surgically implanted into said patient; or at the site of a surgically-created anastomosis of said patient.

10. A pharmaceutical composition comprising a compound of formula I: 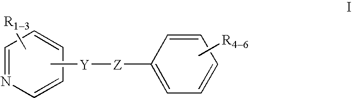
wherein,
Y is selected from CH₂, C(CH₃), CH₂CH₂, CH₂CH₂CH₂, CH₂CH═CH and CH₂CH C;
Z is selected from S, SO, SO₂, O, and NR⁷;
each of R₁, R₂, R₃, R₄, R₅, and R₆ are, independently, selected from H, halide, CF₃, C₁₋₃ alkyl, C₁₋₃ alkoxy, OH, SH, NO₂, CO₂H, SO₃H, and CN; and
R⁷ is selected from H and C₁₋₃ alkyl;
and a pharmaceutically acceptable excipient.

11. The composition of claim 10, wherein the structure of said compound is selected from:

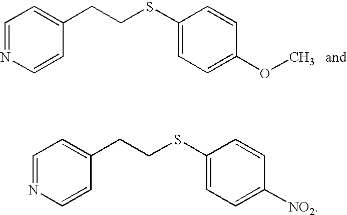

12. A method for determining whether a candidate gene is a component of or affects a molecular pathway involved in vasculogenesis, said method comprising (i) treating a gridlock mutant embryo with a gridlock suppressor, and (ii) determining the effect of said suppressor on the expression of said gene, wherein detection of an altered level of expression of said gene, relative to the level in an untreated gridlock mutant embryo, indicates that the candidate gene is a component of or affects a molecular pathway involved in vasculogenesis.

13. The method of claim 12, wherein the expression of said gene is analyzed by in situ hybridization or real-time polymerase chain reaction.

14. A method for identifying a gene in a molecular pathway involved in vasculogenesis, said method comprising (i) treating a gridlock mutant embryo with a gridlock suppressor, (ii) extracting RNA from the treated embryo, (iii) reverse transcribing the extracted RNA into cDNA, (iv) contacting the cDNA with an array comprising an oligonucleotide library, and (v) identifying any genes corresponding to oligonucleotides of the array to which the cDNA from the treated embryo binds in a manner that is different from cDNA obtained from an untreated gridlock mutant embryo.

15. A method for identifying a component of a molecular pathway involved in vasculogenesis, said method comprising (i) contacting a preparation comprising a candidate component with a matrix comprising a gridlock suppressor, and (ii) identifying molecules that specifically bind to the gridlock suppressor.

16. The method of claim 12, wherein said gridlock suppressor is within formula (I): 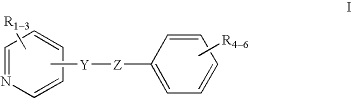
wherein,
Y is selected from CH₂, C(CH₃), CH₂CH₂, CH₂CH₂CH₂, CH₂CH═CH, and CH₂CH C;
Z is selected from S, SO, SO₂, O, and NR⁷;
each of R₁, R₂, R₃, R₄, R₅, and R₆ are, independently, selected from H, halide, CF₃, C₁₋₃ alkyl, C₁₋₃ alkoxy, OH, SH, NO₂, CO₂H, SO₃H, and CN; and
R⁷ is selected from H and C₁₋₃ alkyl.

17. The method of claim 16, wherein the structure of said gridlock suppressor is selected from:

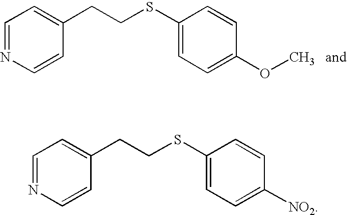

18. The method of claim 14, wherein said gridlock suppressor is within formula (I): 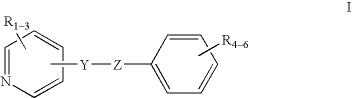
wherein,
Y is selected from CH₂, C(CH₃), CH₂CH₂, CH₂CH₂CH₂, CH₂CH═CH, and CH₂CH C;
Z is selected from S, SO, SO₂, O, and NR⁷;
each of R₁, R₂, R₃, R₄, R₅, and R₆ are, independently, selected from H, halide, CF₃, C₁₋₃ alkyl, C₁₋₃ alkoxy, OH, SH, NO₂, CO₂H, SO₃H, and CN; and
R⁷ is selected from H and C₁₋₃ alkyl.

19. The method of claim 18, wherein the structure of said gridlock suppressor is selected from:

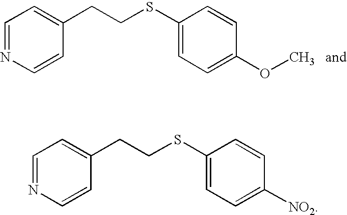

20. The method of claim 15, wherein said gridlock suppressor is within formula (I): 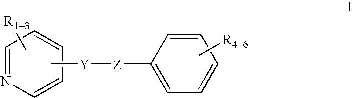
wherein,
Y is selected from CH₂, C(CH₃), CH₂CH₂, CH₂CH₂CH₂, CH₂CH═CH, and CH₂CH C;
Z is selected from S, SO, SO₂, O, and NR⁷;
each of R₁, R₂, R₃, R₄, R₅, and R₆ are, independently, selected from H, halide, CF₃, C₁₋₃ alkyl, C₁₋₃ alkoxy, OH, SH, NO₂, CO₂H, SO₃H, and CN; and
R⁷ is selected from H and C₁₋₃ alkyl.

21. The method of claim 20, wherein the structure of said gridlock suppressor is selected from: [chemical structure]
